(19) United States
(12) Patent Application Publication
(10) Pub. No.: US 2003/0032972 A1
(43) Pub. Date: Feb. 13, 2003

(54) Transmyocardial revascularization gun

(76) Inventor: Peter L. Birnbaum, Fresno, CA (US)

Correspondence Address: Richard A. Ryan, RYAN & ENGNATH, Suite 104, 8469 N. Millbrook, Fresno, CA, 93720, US

(21) Appl. No.: 10/217,169

(22) Filed: Aug. 12, 2002

Related U.S. Application Data
(60) Provisional application No. 60/311,627, filed on Aug. 13, 2001.

(51) Int. Cl. A61B 17/32

(52) U.S. Cl. 606/184

(57) ABSTRACT

A hand-held transmyocardial revascularization gun for ablatively creating channels in tissue, such as heart muscle. The gun is configured with a barrel having a chamber therein for enclosing a probing mechanism and a coring mechanism. The barrel has an opening to permit the surgeon to utilize a finger to slidably move the probing mechanism into the tissue to verify the position is suitable for channel formation. A trigger attached to the gun is used to extend the coring mechanism into the tissue to core out a section thereof for the channel. The opening allows the surgeon to finely control the movement of the probe mechanism to provide tactile feed back, while advancing the probe through the tissue, thus avoiding damage to the internal tissue structures. Stents with or without angiogenic agents thereon can be inserted into the channels to promote vascular or heart cell growth.

### CROSS-REFERENCE TO RELATED APPLICATIONS

This application claims the benefit of U.S. Provisional Application No. 60/311,627 filed Aug. 13, 2001.

### BACKGROUND OF THE INVENTION

A. Field of the Invention

The field of the present invention relates generally to apparatuses and methods for creating vascular channels in the heart and other tissue to enhance blood flow in, and hence healing of, the tissue. More particularly, the present invention relates to such apparatuses and methods that create vascular channels without damaging the tissue along the interior wall of the channel.

B. Background

Heart disease is a major medical concern in the United States and the rest of the world. Many deaths and debilitating injuries result from heart attacks and strokes each year. One of the major heart problems is coronary artery disease that results from the blockage of the blood vessels (coronary arteries) that feed oxygen and blood to the muscle tissue that makes up the heart. The coronary arteries become blocked from a build-up of fats and plaques in the arteries. When the coronary arteries are severely narrowed or completely blocked, as shown in FIG. 1, the heart muscle cells in the part of the heart that was receiving the blood flow dies. This can lead to a heart attack. Often warning signs exist prior to the heart attack that can indicate there is a problem. One such warning sign is known as angina, which is a transient pain or discomfort primarily in the chest area due to a temporary imbalance between the demand for oxygen by the heart muscle and the ability of the coronary arteries to supply enough blood to meet that demand.

The typical medical procedure for treating someone with severe or complete blockage has been to utilize bypass surgery or angioplasty. Bypass surgery involves the removal of a healthy blood vessel (i.e., either an artery or a vein) from one part of the body and attachment of the blood vessel to the heart so as to detour or bypass around the blocked portion of the coronary artery. Blood then flows in the coronary artery, through the bypass section, to nourish the heart with oxygen. Often, a person needing bypass surgery will undergo more than one bypass at the same time. Angioplasty utilizes a balloon like device to push the fatty plaque back against the artery wall to make more room for blood to flow through the artery so as to increase blood flow to the heart. Typically, the balloon is attached on the end of a catheter that is inserted into the femoral artery in the upper thigh or groin area and then snaked up through the artery to the heart. Once at the location of the blockage, the balloon is inflated to clear the artery. Often, a stent formed from a wire meshed tube is then inserted into the artery to hold it open.

For various reasons, such as too much of the coronary artery is blocked or the patient cannot handle the stress of major surgery, neither the bypass surgery nor angioplasty procedures are appropriate for a number of people. If medication does not help, the only recourse has been to have a heart transplant. In recent years, doctors have developed new procedures, such as transmyocardial revascularization (hereinafter referred to as “TMR”), to alleviate the problems associated with blocked coronary arteries and new technologies to stimulate the patient's own body to grow new blood vessels so as to improve blood flow to the heart (interventional angiogenesis). In the typical prior art TMR procedure, the surgeon makes a small opening into the patient's chest and places a specially manufactured laser against the exposed heart. As illustrated in FIG. 2, the surgeon utilizes the laser to “drill” a series of very small holes through the heart muscle, the myocardium, into the pumping chamber inside the heart. Typically, the surgeon will drill between twenty and forty 1 mm holes with the laser apparatus. These holes, commonly referred to as channels, open a pathway from the heart chamber to the muscle around the heart to allow oxygen-filled blood to flow into the heart muscle, a process which is similar to the way certain reptiles (including snakes and alligators) deliver blood/oxygen to their heart. In addition, it is believed that the creation of the channels promotes the body's release of angiogenic agents that encourages the growth of small blood vessels (i.e., angiogenic capillary formation). After the surgery, the exterior entrance is blocked to form a clot and/or stitched to close the opening.

Although the laser TMR procedure has had some substantial success, it does have some significant limitations. One of these is the fact that the laser, while cutting the channel, also damages the heart muscle around the channel (i.e., vaporization creating a zone of necrosis), thereby limiting the amount of blood that can flow into the heart muscle through the channel wall. Often, this may altogether prevent blood flow through the channel. Another limitation with laser TMR is that the laser must be very carefully utilized to prevent damaging the heart wall. Because of the risk of damage, the laser is only fired during the second phase (the systole phase) of the heartbeat cycle when the heart wall is the thickest, as measured by an electrocardiogram connected to the patient. Another limitation is the cost of the laser equipment itself, which can be as much as $300,000 or more, and the special facilities and safety measures that must be employed. The cost and required facilities makes it very difficult for most small hospitals to provide the laser TMR procedure.

As an alternative to the laser TMR device, U.S. Pat. No. 6,306,125 to Parker, et al. describes an angiogenic implant delivery system and method that is used to non-ablatively (i.e., by not removing any portion of the subject tissue) create a channel in the heart muscle and introduce a biodegradable implant into the channel to stimulate production of angiogenic agents by the heart. A method of enhancing blood flow in tissue, which can utilize the above TMR device is described in 6,263,880 to Parker, et al. Theses patents, the full disclosures of which are incorporated herein by reference, describe an implant delivery device for use with the patented method of enhancing blood flow that has an actuator-driven section of hypodermic tubing which is configured to pierce the heart tissue while carrying and then placing the implant into the channel created by the tubing (i.e., by pushing tissue aside). The actuator, such as a double acting pneumatic cylinder, rapidly drives the tubing into the heart tissue while the heart is at rest. The TMR device can include a depth gauge for controlling the distance in which the tubing is driven into the heart tissue. The preferred stint enters the channel in a relatively stiff state that softens to form a compliant hydrodynamic polymer gel that can absorb the angiogenic agents produced by the tissue and, over time, reintroduce those agents into the tissue to prolong and enhance the angiogenic response and revascularization of the tissue. In an alternative embodiment, the splint itself is configured to pierce the heart tissue.

A disadvantage of the actuator-driven implant delivery system of the Parker, et al. patents is that the device rapidly drives the hypodermic tubing into the heart tissue independent of any tactile feedback for or direct control by the surgeon. As is known in the art, any resistence against entry of the tubing into the tissue could indicate the presence of an obstruction that may not be desirable to pass through or to place an implant into the channel created through the obstruction. As with the laser TMR, the use of an actuator, such as the double acting pneumatic cylinder of the preferred embodiment, removes the control away from the surgeon once the actuator is activated, thereby preventing the surgeon from stopping the penetration and insertion if he or she encounters an obstruction. In addition, the device non-ablatively introduces the implant into the heart by merely moving the heart tissue aside, as opposed to actually removing the tissue, as with the laser TMR (which vaporizes the tissue away).

Therefore, what is needed is an TMR apparatus to more efficiently and effectively open channels into the heart while eliminating the risks associated with the use of laser surgery and actuator-driven systems. The preferred apparatus should be easy to use, adaptable to current surgery techniques and significantly less expensive than lasers. To be effective, such an apparatus should be suitable for opening one or more channels into the heart muscle without damaging the walls of the channels so as to allow blood to flow into the heart muscle and allow the surgeon direct control over the channel creation process.

### SUMMARY OF THE INVENTION

The TMR gun of the present invention solves the problems identified above. That is to say, the present invention discloses a new and useful mechanical apparatus for TMR procedures which is relatively inexpensive to manufacture, easy to use and effective at opening channels into the heart muscle. The TMR gun of the present invention simplifies the process of creating such channels and significantly reduces the cost of performing TMR procedures (relative to the cost of the laser machines for laser TMR).

In the preferred embodiment of the present invention, the TMR gun for ablatively forming a channel in a tissue has a barrel with a proximal end and an opposing distal end (relative to the position of the surgeon holding the gun). The distal end of the barrel is shaped to abut the tissue and inside the barrel is a chamber, which can be configured as a longitudinal channel. A probing mechanism for initially penetrating the tissue is slidably disposed in the chamber. The first end of the probing mechanism is shaped and configured to penetrate the tissue and the opposing second end of the probing mechanism is, in the preferred embodiment, configured to wrap or coil around a rotatable shaft at or near the proximal end of the barrel. Also located inside the chamber and disposed at or near the distal end of the barrel is a coring mechanism, such as a core biopsy device, that is configured for coring out a section of the tissue, such as the heart muscle, to form the channel. The preferred embodiment also has a trigger operatively connected to the coring mechanism to move it from a retracted position inside the barrel to an extended position outside the distal end of the barrel so as to core out the channel and a handle for holding the TMR gun.

In the preferred embodiment of the TMR gun of the present invention, the gun further comprises a probe control mechanism to allow the surgeon to receive tactile feedback from the probe so as to selectively control the movement of the probing mechanism while moving the first end of the probing mechanism from a first position in the chamber to a second position extending beyond the distal end of the barrel. The control mechanism can be an opening in the barrel that allows the surgeon to contact the probing mechanism in the chamber and cause the probe mechanism to move in and out of the distal end of the barrel. Preferably, the opening is sized and configured to allow the surgeon to place a finger into the opening to move the probing mechanism from the first position to the second position and back. In another embodiment, the control mechanism can be a wheel member disposed in the barrel, with the wheel member being in operative engagement with the probing mechanism to move the probing mechanism from the first position to the second position.

The probing mechanism of the preferred embodiment is a wire or wire-type component that is slidably disposed inside the chamber. The probe wire is pushed into the tissue by the surgeon's finger movement of the probe wire in the opening. The probe wire moves inside the coring mechanism, such that the coring mechanism cores out and removes a portion of the tissue around the probe wire to form the channel. After the probe wire enters the tissue, indicating that there are no obstructions that could be a problem with formation of the channel, the trigger is operated to cause the coring mechanism to core out the channel. Release of the trigger results in the coring mechanism moving back into the distal end of the barrel. The probe wire is then slid back into the chamber by movement of the probe wire in the opening or in conjunction with the coring mechanism.

Accordingly, the primary objective of the present invention is to provide a TMR gun that overcomes the disadvantages associated with laser TMR and with non-ablative TMR devices that are used to form a channel in a tissue, such as heart muscle.

It is also an important objective of the present invention to provide a TMR gun that creates channels into the heart without damaging the channel walls to allow blood to flow from the interior chamber of the heart into the heart muscle.

It is also an important objective of the present invention to provide a TMR gun that is an easily operated, hand-held device that does not require source of electrical or pneumatic power.

It is also an important objective of the present invention to provide a TMR gun comprising a probing mechanism and a coring mechanism in a housing having a chamber or channel inside for slidable movement of the probing mechanism to facilitate safe and effective coring of a tissue.

The above and other objectives of the present invention will be explained in greater detail by reference to the attached figures and the description of the preferred embodiment which follows. As set forth herein, the present invention resides in the novel features of form, construction, mode of operation and combination of processes presently described and understood by the claims.

### BRIEF DESCRIPTION OF THE DRAWINGS

In the drawings which illustrate the best modes presently contemplated for carrying out the present invention:

FIG. 1 illustrates a heart having both a partial and complete coronary artery blockage;

FIG. 2 illustrates the prior art method and device of utilizing lasers to perform the TMR procedure;

FIG. 3 is a perspective view of the TMR gun of the present invention showing the various components thereof in a non-use condition (i.e., retracted into the barrel);

FIG. 4 is a cut-away side view of the TMR gun of the present invention showing the probe wire extended beyond the distal end of the gun;

FIG. 5 is a cut-away side view of the TMR gun of the present invention showing the probe wire and the coring mechanism extended beyond the distal end of the gun;

FIG. 6 is a top view of the TMR gun of the present invention with the coring apparatus shown partially deployed;

FIG. 7 is an end view of the distal end of the TMR gun of the present invention;

FIG. 8 is a cut-away section of the heart muscle showing a channel formed therein by the TMR gun of the present invention and a stent to be placed in the channel;

FIG. 9 is a cut-away section of the heart muscle showing a stent positioned in the channel formed by the TMR gun of the present invention;

FIG. 10 is a photomicrograph of a myocardial channel created with a laser TMR device;

FIG. 11 is a photomicrograph of a myocardial channel created with a prototype TMR gun of the present invention;

FIG. 12 is a top view of an alternative embodiment of the present invention showing use of a wheel member to control movement of the probe wire.

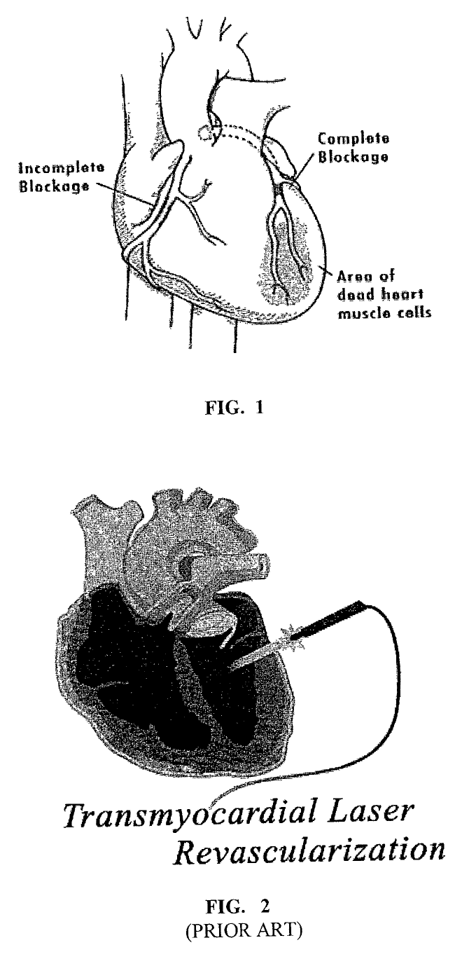

### DETAILED DESCRIPTION OF THE PREFERRED EMBODIMENTS

With reference to the figures where like elements have been given like numerical designations to facilitate the reader's understanding of the present invention, and particularly with reference to the embodiment of the present invention illustrated in FIGS. 3 through 7, the preferred embodiments of the present invention are set forth below. In the preferred embodiment of the present invention, the TMR gun of the present invention, identified generally as 10, is a small hand-held gun type of instrument having a barrel 12, handle 14 and a trigger 16. As set forth below and in the figures, handle 14 and trigger 16 project generally outwardly from the barrel and trigger 16 can be of the type that is operated by pulling or squeezing trigger 16 towards handle 14 with pressure supplied by one or more of the surgeon's fingers as the palm of the surgeon's hand is held against handle 14.

Barrel 12 should be made out of plastic or other lightweight, strong and chemical resistant material that is configured to provide a surgical tool which is easy to hold for extended periods (i.e., during a length surgery) and which is easy to clean and sterilize. Many other materials may also be suitable for gun 10, including various metals or composite materials, including stainless steel and carbon fiber materials. The TMR gun can be made to be disposable or it can be made to be cleanable by presently available cleaning methods. In the embodiment of the present invention shown in the figures, barrel 12 is shown to comprise a generally cylindrical shape having opposing ends, proximal end 18 and distal end 20 (designated as such relative to the position of the surgeon when performing the TMR procedures described herein). In one embodiment, the barrel has an outer diameter of one-half inches and a length of six inches. However, barrel 12 of the TMR gun 10 of the present invention can be configured into a variety of sizes and shapes, as may be suitable for comfortably holding the gun 10 and for desirable aesthetic purposes. Distal end 20 should be configured to abut or be engagable with the tissue that is to be treated. While a generally planar distal end 20 is typically suitable for most TMR procedures, distal end 20 can be contoured or otherwise configured to more preferentially abut the tissue that is to be treated.

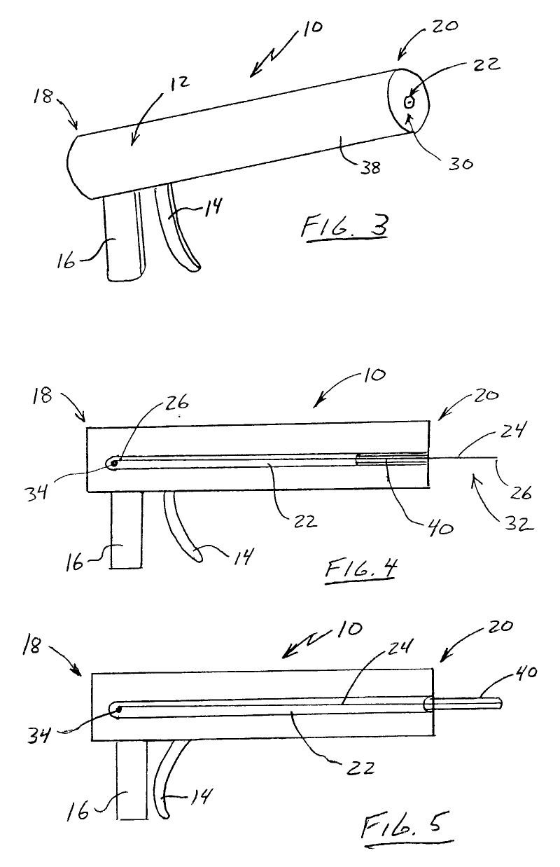

The interior of barrel 12 comprises a chamber 22, which can be in the form of an elongated channel for all or part of the length of barrel 12, in which is located and through which travels a probing mechanism, such as thin, stiff probe wire 24 having a first end 26 and second end 28. The first end 26 of probe wire 24 should be appropriately configured, such as by being cut at an angle, to allow probe wire 24 to efficiently penetrate the tissue. As explained more fully below and shown in FIGS. 3 and 4, probe wire 24 should be sufficiently stiff to allow the surgeon to slide the probe wire 24 inside chamber 22 so as to cause probe wire 24 to move from a first position 30 where first end 26 is inside barrel 12 to a second position 32 where first end 26 extends beyond the distal end 20 of barrel 12. In one embodiment, probe wire 24 has a diameter of one mm and has a length approximately equal to the full length of barrel 12, although it would not be necessary for it to be that long. In a preferred embodiment, the second end 28 of probing mechanism wraps or coils around a rotatable shaft 34 positioned at or near the proximal end 18 of barrel 12 as the first end 26 of probe wire 24 moves back from the second position 32 to the first position 30. In an alternative embodiment, the probing mechanism can be a stiff wire or needle that merely moves, in a generally longitudinal direction, inside chamber 22 but does not coil around any shaft, such that shaft 34 is not needed. In this embodiment, the probe wire 24 or other wire-like member could frictionally engage the walls of chamber 22 to allow the surgeon to maintain sufficient control over the movement of probe wire 24. In the preferred embodiment, when wire probe 24 is not being used for a TMR procedure it is preferred that probe wire 24 be fully contained inside barrel 12 to protect first end 26 from damage. The probing mechanism should be made out of a material suitable for use to pierce human tissue, including the heart. Such materials include stainless steel, carbon fiber and like materials.

The TMR gun 10 of the present invention also includes a mechanism for controlling or allowing control of the movement of the probing mechanism (i.e., probe wire 24). In the preferred embodiment, as best shown in FIG. 6, the barrel includes an opening 36 in a side 38 of barrel 12 that is in communication with chamber 22 to expose a section of probe wire 24. Also in the preferred embodiment, opening 36 is sized and configured to allow the surgeon to place his or her finger inside opening 36 to push against and slide probe wire 24 so as to cause first end 26 of probe wire 24 to move from first position 30 to second position 32 to penetrate the tissue or to move from second position 32 to first position to retract probe wire 24 after piercing the tissue. When the surgeon places his or her finger inside opening 36 and pushes against probe wire 24 to cause it to penetrate the tissue, any resistance against probe wire 24 will be felt by the surgeon, providing the surgeon with nearly instant tactile feedback on his or her progress. Depending on the nature of the resistance, the surgeon can decide to cease any further penetration into the tissue and either further evaluate that area of the tissue or move to a different penetration point. For instance, the heart is made up of internal cardiac structures that could be damaged by further penetration. Use of opening 36, such as a generally square or rectangular opening that is approximately three inches long, allows the surgeon substantially more feel of what is happening and control over the penetration than is possible with presently available mechanical or electro-mechanical devices.

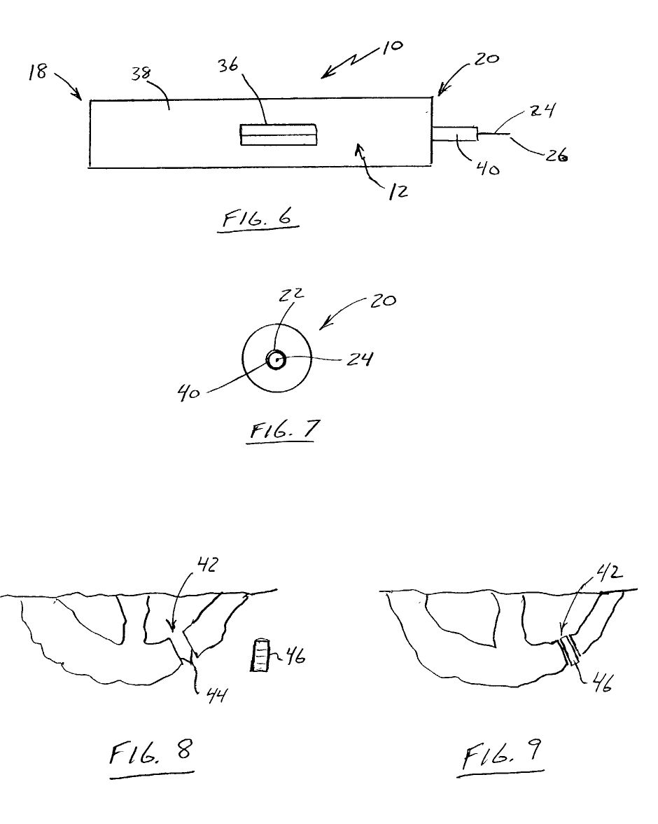

Operatively connected to trigger 16 is a coring mechanism 40, such as those used for performing biopsy cores, suitable for coring a 1 mm section (or other desirable size of core) from the tissue (i.e., the heart muscle). As is well known, core biopsy devices generally comprise two sharp metal projections, one orientated adjacent to the other. A typical size for coring mechanism 40 is an outside diameter of approximately 1.3 mm and a length of 30 mm. As with probe wire 24, coring mechanism 40 should be made out of a material suitable for penetrating and coring out a section of human tissue. As shown in the figures, coring mechanism 40 is located at or near the distal end 20 of barrel 12 and configured for probe wire 24 to travel through coring mechanism 40 (as best shown in FIG. 7). In use, the coring instrument is advanced to abut the tissue to be cored and then fired, causing one of the metal projections to advance relative to the other, thus cutting out a core of tissue. In its non-use condition, coring mechanism 40 is retracted into barrel 12 such that the distal end 20 of gun 10 is generally planar. Coring mechanism 40 is operatively connected to trigger 16 such that when trigger 16 is activated, coring mechanism 40 rapidly extends beyond distal end 20 of barrel 12 to core out a section of the heart muscle (similar to the way a biopsy is performed). Upon release of trigger 16, coring mechanism 40 retracts back into barrel 12.

In use, the surgeon exposes the patient's heart or other tissue and, with both the probe wire 24 and coring mechanism 40 in their retracted positions, as shown in FIG. 3, places the distal end of barrel 12 against the portion of the heart muscle or other tissue that is to receive the TMR procedure. Once in position, the surgeon places his or her finger in opening 36 to push or slide the probe wire 24 forward using his or her finger in the opening 36, as shown in FIG. 4. Pushing on probe wire 24 by the surgeon causes probe wire 24 to extend beyond distal end 20 of barrel 12, causing the probe wire 24 to enter into the heart muscle and penetrate it to the chamber inside. By utilizing a finger to control the forward movement of the probe wire 24, the surgeon can receive tactile feedback so as to finely control the speed of the probe wire 24 and the amount which it penetrates the heart muscle. Once probe wire 24 is in its proper place and has penetrated the heart muscle without damage, the surgeon activates coring mechanism 40 by pulling trigger 16 towards handle 14. When activated, coring mechanism 40 extends beyond the distal end 20 of barrel 12, as shown in FIG. 5, to core out a section (i.e., a 1 mm diameter core) of the heart tissue and form channel 42. Upon release of trigger 16, the coring mechanism 40 retracts. The surgeon then backs probe wire 24 out by using his or her finger in the opening 36 to slide probe wire 24 backward into barrel 12, causing it to wrap or coil around shaft 34 (if it is used). When the surgeon removes gun 10 from the heart, a channel 42 (shown in FIGS. 8 and 11) is left in the heart, the outside end of which is typically closed utilizing currently available procedures (i.e., holding a finger against the hole and/or using sutures).

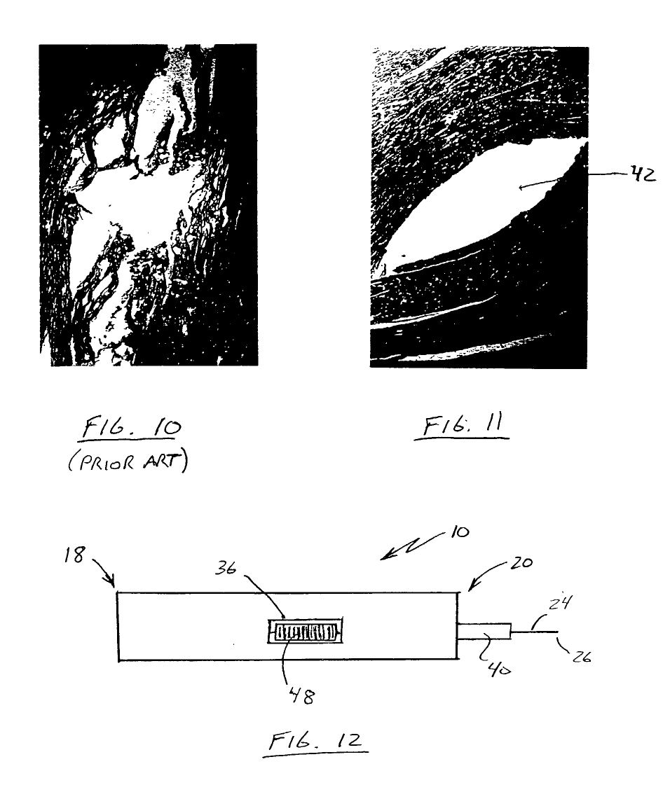

Use of TMR gun 10 having a coring mechanism 40 instead of the laser to ablatively create channel 42 has certain significant advantages, including leaving a channel 42 that is much less likely to have any traumatized tissue along the channel wall 44 or any vaporization that results in a zone of necrosis. FIGS. 10 and 11 are photomicrographs of myocardial channels created with a prior art laser TMR device (FIG. 10) and a prototype of the TMR gun 10 of the present invention. As shown in these figures, the channel 42 in FIG. 11 is cleaner and much more open. Because the channels 42 in the heart formed with the TMR gun 10 of the present invention are much more open, the procedure will improve the flow of blood and oxygen to the heart muscle, which is more likely to encourage growth of small blood vessels and improve the patient's health. In addition, the cleaner channel 42 will better facilitate the use of a biodegradable, porous stent 46 (shown in FIGS. 8 and 9) that can be inserted into the channel 42 created by the TMR gun 10 of the present invention. Stent 46 can be coated or otherwise contain cell cultures or other substances that may promote heart cell or vascular growth or repair to further improve the health of the heart. Another advantage of gun 10 is that the surgeon controls the entry of probe wire 24 with his or her finger, therefore, the surgeon can feel any obstruction or other problems with the entry of probe wire 24 before it fully enters the heart muscle, something which cannot be done with use of the laser to create channel 42 or with the non-ablative device of Parker, et al. Yet another advantage of the TMR gun 10 is that is relatively inexpensive to make, particularly relative to the laser, which should allow many more facilities and doctors to offer the TMR procedure.

Various modifications can be made to the TMR gun 10 of the present invention. One such modification, shown in FIG. 10, is to utilize a wheel member 48 placed in opening 36 and operatively engaged with probe wire 24 so the surgeon can push or roll his or her finger against the wheel member 48 to move the probe wire 24 forward (out of the barrel 12) or backward (back into barrel 12). The use of wheel member 48 may make it easer for the surgeon to move probe wire 24 in and out of gun 10 and still provide the surgeon with the feel and control of probe wire 24 necessary to prevent damaging the heart muscle. Other types of control devices, as are known in the industry, can be used to control the forward and backward movement of probe wire 24, including various mechanical and electronic devices. Another modification to gun 10 is the use of a different trigger mechanism to activate the coring mechanism 40, rather than squeezing trigger 16 as described above. Trigger 16 can be a variety of mechanical and/or electrical devices that allow the surgeon to selectively cause coring mechanism 40 to penetrate and core the heart tissue to form channel 42. For instance, trigger 16 can be an electronic device that the surgeon merely pushes to cause the coring device to rapidly extend from the distal end 20 of gun 10 into the heart tissue. Trigger 16 and coring mechanism 40 can be jointly configured such that the coring mechanism 40 automatically retracts upon coring the heart tissue. In addition, the retraction of the coring mechanism 40 can be configured so that it also retracts probe wire 24 when the core is done, such that the retraction of probe wire 24 and coring mechanism 40 can occur with one motion, either simultaneously or sequentially, thereby avoiding the need of having to manually retract probe wire 24.

The TMR gun 20 of the present invention can also be configured to be able to insert any desired stents or implants 46, with or without any angionic agents thereon to promote vascular growth and/or heart cell growth. Such agents include myocyte cell culture and/or vascular endothelialgrowth factor (VEGF), as well as other known agents. The Parker, et al. patents referenced herein, describe the operation and configuration of implants and the use of agents and growth factors on or in the implant.

While there are shown and described herein certain specific alternative forms of the invention, it will be readily apparent to those skilled in the art that the invention is not so limited, but is susceptible to various modifications and rearrangements in design and materials without departing from the spirit and scope of the invention. In particular, it should be noted that the present invention is subject to modification with regard to the dimensional relationships set forth herein and modifications in assembly, materials, size, shape and use.

What is claimed is:

1. A transmyocardial revascularization gun for ablatively forming a channel in a tissue, comprising:
a barrel having a proximal end and an opposing distal end, said distal end shaped to abut the tissue;
a chamber disposed in said barrel;
probing means disposed in said chamber for penetrating the tissue, said probing means having a first end and an opposing second end, said first end of said probing means shaped and configured to penetrate the tissue;
coring means at said distal end of said barrel for coring out a section of the tissue to form the channel; and
a trigger operatively connected to said coring means to move said coring means from a retracted position to an extended position so as to core out the channel.

2. The transmyocardial revascularization gun according to claim 1 further comprising probe control means on said barrel to allow for selectively controlling the movement of said probing means to move said first end of said probing means from a first position in said chamber to a second position extending beyond said distal end of said barrel.

3. The transmyocardial revascularization gun according to claim 2, wherein said control means comprises an opening in said barrel allowing contact with said probing means in said chamber.

4. The transmyocardial revascularization gun according to claim 3, wherein said opening is sized and configured to allow a surgeon to place a finger into said opening to move said probing means from said first position to said second position.

5. The transmyocardial revascularization gun according to claim 2, wherein said control means comprises a wheel member disposed in said barrel, said wheel member in operative engagement with said probing means to move said probing means from said first position to said second position.

6. The transmyocardial revascularization gun according to claim 1 further comprising a rotatable shaft disposed at said second end of said barrel, said probing means configured to wrap around said rotatable shaft as said first end of said probing means moves from said second position to said first position.

7. The transmyocardial revascularization gun according to claim 1, wherein said probing means is a wire.

8. The transmyocardial revascularization gun according to claim 1, wherein said coring means is disposed in said chamber and said probing means is configured to move through said coring means.

9. The transmyocardial revascularization gun according to claim 1, wherein said trigger projects outwardly from said barrel.

10. The transmyocardial revascularization gun according to claim 9 further comprising a handle disposed proximally of said trigger, said trigger configured to be movable towards said handle so as to cause said coring means to move from said retracted position to said extended position.

11. The transmyocardial revascularization gun according to claim 1 further comprising a handle projecting outwardly from said barrel, said handle configured to be held by a surgeon while utilizing the gun to form the channel in the tissue.

12. A transmyocardial revascularization gun for ablatively forming a channel in a tissue, comprising:
a barrel having a proximal end and an opposing distal end, said distal end shaped to abut the tissue;
a chamber disposed in said barrel;
a probe wire slidably disposed in said chamber, said probe wire having a first end and an opposing second end, said first end of said probe wire shaped and configured to penetrate the tissue;
an opening in said barrel to allow a surgeon to contact said probe wire in said chamber so as to selectively control the movement of said first end of said probe wire from a first position in said barrel to a second position extending beyond said distal end of said barrel;
coring means disposed in said chamber at said distal end of said barrel for coring out a section of the tissue to form the channel; and
a trigger operatively connected to said coring means to move said coring means from a retracted position to an extended position so as to core out the channel.

13. The transmyocardial revascularization gun according to claim 12, wherein said opening is sized and configured to allow a surgeon to place a finger into said opening to move said probe wire from said first position to said second position.

14. The transmyocardial revascularization gun according to claim 12 further comprising a wheel member disposed in said opening, said wheel member in operative engagement with said probe wire to move said probe wire from said first position to said second position.

15. The transmyocardial revascularization gun according to claim 1 further comprising a rotatable shaft disposed at said second end of said barrel, said probe wire configured to wrap around said rotatable shaft as said first end of said probe wire moves from said second position to said first position.

16. The transmyocardial revascularization gun according to claim 1, wherein probe wire is configured to slidably move through said coring means.

17. The transmyocardial revascularization gun according to claim 1 further comprising a handle projecting outwardly from said barrel, said handle configured to be held by a surgeon while utilizing the gun to form the channel in the tissue.

18. A transmyocardial revascularization gun for ablatively forming a channel in a tissue, comprising:
a barrel having a proximal end and an opposing distal end, said distal end shaped to abut the tissue;
a chamber disposed in said barrel;
a probe wire slidably disposed in said chamber, said probe wire having a first end and an opposing second end, said first end of said probe wire shaped and configured to penetrate the tissue;
an opening in said barrel to allow a surgeon to contact said probe wire in said chamber so as to selectively control the movement of said probe wire from a first position in said chamber to a second position extending beyond said distal end of said barrel;
a core biopsy device disposed in said chamber at said distal end of said barrel, said probe wire positioned to slidably move through said core biopsy device; and
a trigger operatively connected to said core biopsy device to move said core biopsy device from a retracted position to an extended position so as to core out the channel.

19. The transmyocardial revascularization gun according to claim 18 further comprising a wheel member disposed in said opening, said wheel member in operative engagement with said probe wire to move said probe wire from said first position to said second position.

20. The transmyocardial revascularization gun according to claim 18 further comprising a rotatable shaft disposed at said second end of said barrel, said probe wire configured to wrap around said rotatable shaft as said first end of said probe wire moves from said second position to said first position.
